(19) United States
(12) Patent Application Publication
(10) Pub. No.: US 2021/0059821 A1
(43) Pub. Date: Mar. 4, 2021

(54) IMPLANTABLE CONTAINMENT APPARATUS

(71) Applicant: Michael Craig McMains, Carmel, IN (US)

(72) Inventor: Michael Craig McMains, Carmel, IN (US)

(21) Appl. No.: 17/007,214

(22) Filed: Aug. 31, 2020

Related U.S. Application Data
(60) Provisional application No. 62/893,366, filed on Aug. 29, 2019.

(51) Int. Cl. A61F 2/28; A61F 2/44; A61F 2/46

(52) U.S. Cl. CPC A61F 2/2846; A61F 2002/30062; A61F 2/4601; A61F 2/4455

(57) ABSTRACT

An implant containment apparatus for use with an implant device that defines a cavity that contains a bone graft material, and an implant tool selectively attached to the implant device, the implant containment includes a sack that defines a compartment and an opening that provides access to the compartment, where the compartment is adapted to contain the implant device with bone graft in the cavity and where the opening is adapted to allow the implant tool to extend out of the sack when the implant device is in the compartment, and a pre-attached closing member surrounding the opening adapted to selectively vary the size of the opening between a size that tightly fits around the implant tool and a size that closes the opening.

### CROSS-REFERENCE TO RELATED APPLICATIONS

This application claims the benefit of U.S. Provisional Application No. 62/893,366, filed Aug. 29, 2019, which is hereby incorporated by reference.

### BACKGROUND

This disclosure is in the field of implantable devices for containing implanted materials such as bone cement.

### BRIEF DESCRIPTION OF THE DRAWINGS

FIG. 1 depicts a side view of a sack, an interbody cage and an insertion handle for the interbody cage.

FIG. 2 depicts a side view of the FIG. 1 sack with the interbody cage contained within the sack and with a closing member closing an opening in the sack about the FIG. 1 insertion handle.

FIG. 3 depicts a side view of the FIG. 1 sack with the interbody cage contained within the sack and with the closing member completely closing the opening in the sack.

FIG. 4 depicts a method of using the disclosed implantable containment apparatus.

### DESCRIPTION OF THE ILLUSTRATED EMBODIMENTS

For the purposes of promoting an understanding of the principles of what is claimed, reference will now be made to embodiments illustrated in the drawings and specific language will be used to describe the same. It will nevertheless be understood that no limitation of the scope of the claims is thereby intended. Any alterations and further modifications in the illustrated device, and any further applications of the principles disclosed and illustrated herein are contemplated as would normally occur to one skilled in the art to which the disclosure relates.

With respect to the specification and claims, it should be noted that the singular forms “a”, “an”, “the”, and the like include plural references unless expressly discussed otherwise. As an illustration, references to “a device” or “the device” include one or more of such devices and equivalents thereof. It also should be noted that directional terms, such as “up”, “down”, “top”, “bottom”, “upper”, “lower”, “proximal”, “distal”, and the like, are used herein solely for the convenience of the reader in order to aid in the reader's understanding of the illustrated embodiments, and it is not the intent that the use of these directional terms in any manner limit the described, illustrated, and/or claimed features to a specific direction and/or orientation.

Bone, as used herein, refers to bone that is cortical, cancellous or cortico-cancellous of autogenous, allogenic, xenogenic, or transgenic origin.

Some implants may include voids or cavities that are optionally filled with a graft material to promote bone ingrowth into the implant to help fuse the implant in position relative to surround bone. For example, in a spinal cage implant positioned between adjacent vertibre in a spinal fusion procedure. Generally, the voids or cavities in the implant are filled with the graft material outside the body before the implant is positioned in a surgical site, such as between adjacent vertibre. It is important to contain the graft material or other material within the implant during the passage through the patient's body to the final surgical site to prevent contamination of other body tissues with graft material and to maintain the desired volume of graft material in the implant. The delivery system disclosed below addresses this issue by containing the implant and the graft material in a sack.

A delivery system for delivery of a substance in an implant device to a surgical site is disclosed. In various embodiments, the delivery system comprises an implantable sack, for example a mesh bag, having at least one compartment configured to contain an implant device and at least a first substance, for example, a bone graft material such as autograft bone chips, allograft, and/or bone graft substitutes packed in the implant device, for delivery to a surgical site using an implant tool. The sack defines an opening, the opening being configured to receive the implant device packed with bone graft material with the implant device optionally attached to the implant tool for delivery to a surgical site.

The implantable sack also includes a pre-attached closing member surrounding the opening and positioned proximate to the opening. In various embodiments, the pre-attached closing member may be adapted to close around the implant tool to contain the implant and the at least first substance during delivery of the sack and packed implant to the surgical site. The pre-attached closing member further being adapted to permit removal of the implant tool and subsequent closing of the pre-attached closing member to contain the implant and the at least first substance at the surgical site. In other various embodiments, the pre-attached closing member may optionally be closed before implantation to contain the implant and the at least first substance at the surgical site and the closed bag may be manually delivered to a surgical site or an implant tool may optionally be used to deliver the closed bag containing the implant device and first substance to the surgical site.

Generally, the sack may be a single or multi-compartment structure capable of at least partially retaining an implant device and at least a first substance packed in the implant device until the covering and the contained implant device is placed at a surgical site. In some embodiments, the covering may be substantially non-expandable or minimally deformable. In some embodiments, the covering may be a temporary covering wherein the covering is substantially resorbable. For example, in some embodiments, the covering may be formed of a material that is substantially reabsorbed within 2 weeks, within 4 weeks, within 12 weeks, or within other suitable time frame.

In some embodiments, the sack may be used for maintaining materials (including the substance contained in the sack) in spatial proximity to one another, possibly to provide a synergistic effect. In some embodiments, the sack may be used for delivery through a limited opening, such as in minimally invasive surgery or mini-open access.

In various embodiments, the covering contains a substance or material such as a graft material. The covering limits, and in some embodiments eliminates, migration of the graft material. The sack, with contained substance and implant, may be configured to conform to surrounding bony contours or implant space. In some embodiments, the sack provides a pathway for healing/cell penetration and tissue ingrowth. Thus, the sack may facilitate transfer or diffusion of materials into and out of the sack. For example, the sack may facilitate diffusion of a substance provided within the sack or may facilitate diffusion into the sack of materials in the surgical site, such as cells and tissues, into the sack. The sack may be configured to permit diffusion of some materials while substantially preventing diffusion of other materials. The sack may also be configured to permit mechanical interaction between the implant and the surrounding bony contours.

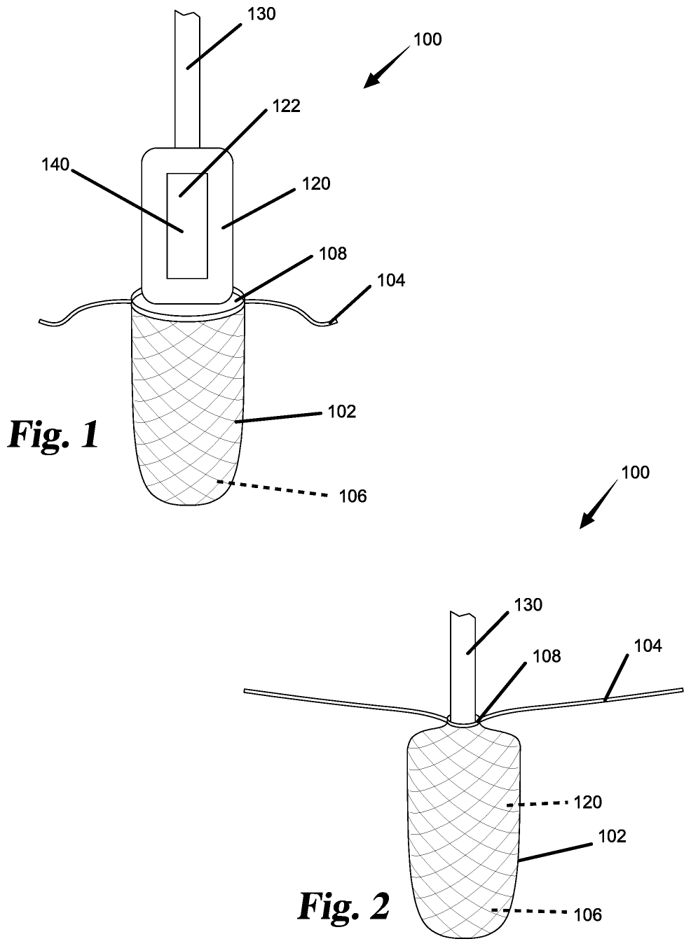

Referring to FIG. 1, implant containment apparatus 100 is illustrated. Implant containment apparatus 100 comprises sack 102 and closing member 104. Implant containment apparatus 100 defines compartment 106 and opening 108 that permits access to compartment 106 from outside of implant containment apparatus 100.

FIG. 1 also illustrates implant device 120 and implant tool 130. In the illustrated embodiment, implant device 120 is an interbody cage adapted for use in spinal fusion procedures to support and maintain spacing between adjacent vertebrae. In the illustrated embodiment, implant device 120 defines cavity 122 that extends through implant device 120. Cavity 122 contains graft material 140, which is adapted to promote bone ingrowth into cavity 122 and through implant device 120. Graft material 140 may comprise material such as patient harvested bone chips, autograft bone chips, allograft, and/or bone graft substitutes. Implant tool 130 is adapted to selectively secure implant device 120 to permit positioning of implant device 120 in a surgical procedure that may have limited address to the insertion site. In the illustrated embodiment, implant tool 130 is connected to implant device 120 by a threaded connection between implant tool 130 and implant device 120. In other embodiments, other connecting techniques can be used, such as a pincer mechanism.

Sack 102 may be a mesh. Such a mesh may optionally be constructed from a suture material. Sack 102 may optionally be constructed from a bioabsorbable material. Sack 102 may optionally be constructed to conform to surrounding bony contours or implant space. In some embodiments, sack 102 may provide a pathway for healing/cell penetration and tissue ingrowth. For example, sack 102 may facilitate diffusion of a substance provided within the sack or may facilitate diffusion into the sack of materials in the surgical site, such as cells and tissues, into the sack. Sack 102 may be configured to permit diffusion of some materials while substantially preventing diffusion of other materials. Sack 102 may optionally be configured to permit mechanical interaction between the implant and the surrounding bony contours.

In the illustrated embodiment, closure member 104 is a length of suture material that extends around opening 108, commonly referred to as a purse string. Closure member 104 may optionally be provided pre-attached to sack 102.

Alternatively, closure member 104 may be created by threading a length of suture to create a desired configuration for closure member 104.

Referring now to FIG. 2, a side view of the FIG. 1 implant containment apparatus 100 is shown with implant device 120 contained within compartment 106 (and not illustrated). Implant tool 130 passes through opening 108. Closing member 104 may optionally be closed about implant tool 130, thereby containing implant device 120 and graft material 140 positioned in cavity 122 within implant containment apparatus 100. This may permit positioning implant device 120 within a patient with implant tool 130 without risking loss of graft material 140 or unintended contamination of a surgical site with graft material 140.

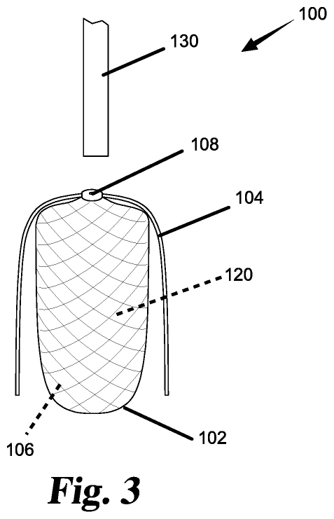

Referring now to FIG. 3, a side view of the FIG. 1 implant containment apparatus 100 is shown with implant tool 130 removed from implant device 120 and closing member 104 completely closed, thereby containing implant device 120 and graft material 140 positioned in cavity 122 within implant containment apparatus 100. This is the configuration of implant containment apparatus 100 and implant device 120 that could be left at a final position within a patient.

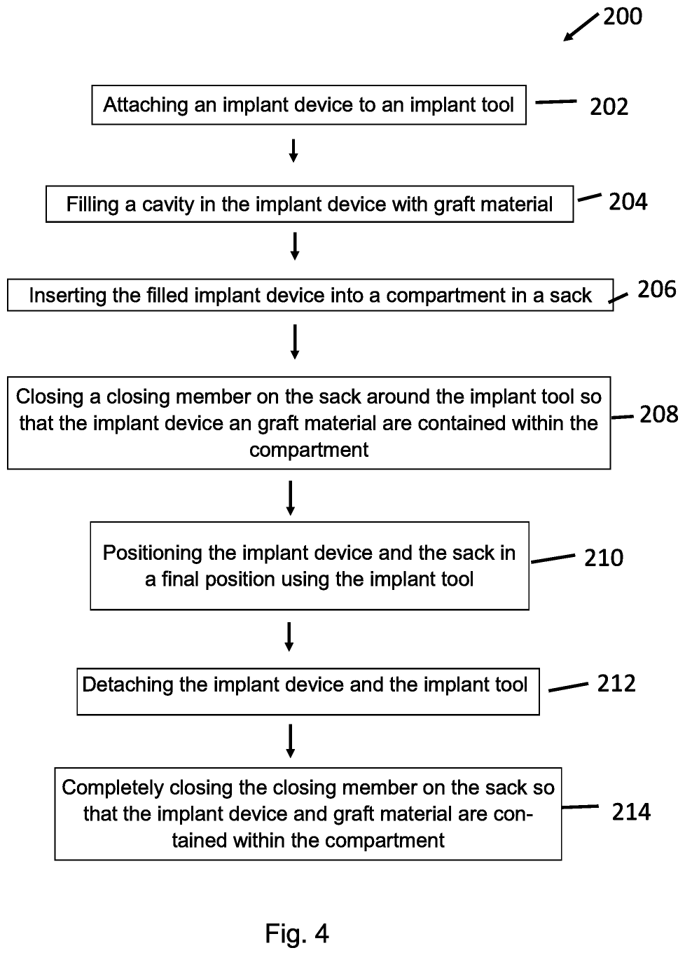

Referring now to FIG. 4, procedure 200 for using implant device 100 is shown. Procedure 202 includes step 202 where implant device 120 is attached to implant tool 130; step 204 where cavity 122 in implant device 120 is filled with graft material 140; step 206 where the filled implant device is inserted into compartment 106 in sack 102; step 208 where closure member 104 on sack 102 is closed around implant tool 130 so that implant device 120 and graft material 140 are contained within compartment 106; step 210 where implant device 120 and sack 102 are positioned in a final position using implant tool 130; step 212 where implant tool 130 is detached from implant device 120; and step 214 where closing member 104 on sack 102 is completely closed so that implant device 120 and graft material 140 are contained within compartment 106.

While the claimed subject matter has been illustrated and described in detail in the drawings and foregoing description, the same is to be considered as illustrative and not restrictive in character. All changes and modifications that come within the spirit of the disclosure are desired to be protected by the claims.

I claim:

1. An implant containment apparatus for use with an implant device that defines a cavity that contains a bone graft material, and an implant tool selectively attached to the implant device, the implant containment apparatus comprising:
a sack defining a compartment and an opening that provides access to the compartment, wherein the compartment is adapted to contain the implant device with bone graft in the cavity and wherein the opening is adapted to allow the implant tool to extend out of the sack when the implant device is in the compartment; and
a pre-attached closing member surrounding the opening adapted to selectively vary the size of the opening between a size that tightly fits around the implant tool and a size that closes the opening.

2. The implant containment apparatus of claim 1, wherein the sack is constructed from a bioabsorbable material.

3. The implant containment apparatus of claim 1, wherein the sack is a mesh.

4. The implant containment apparatus of claim 3, wherein the mesh is constructed from a suture material.

5. The implant containment apparatus of claim 1, wherein the sack is adapted to contain the bone graft material.

6. The implant containment apparatus of claim 1, wherein the pre-attached closing member is a length of suture configured as a purse string.

7. The implant containment apparatus of claim 1, wherein the implant device is an interbody cage adapted for use to fuse two vertebrae together.

8. The implant containment apparatus of claim 7, wherein the interbody cage defines a hole that extends through the interbody cage, wherein the hole is filled with the bone graft material.

9. The implant containment apparatus of claim 1, wherein the closure member is pre-attached to the sack.

10. A method for using the implant containment apparatus of claim 1, the method comprising:
attaching the implant device to the implant tool;
filling the cavity in the implant device with the bone graft material;
after filling the cavity with the bone graft material, inserting the implant device into the compartment in the sack;
closing the pre-attached closing member around the implant tool such that the implant device is contained within the compartment;
with the implant tool, positioning the implant device and the sack in a final position; and
detaching the implant device and the implant tool.

11. The method of claim 10, further comprising: closing the pre-attached closing member to close the opening.

12. The method of claim 11, further comprising: removing excess material from the pre-attached closing member.

13. A system comprising:
an implant device that defines a cavity adapted to contain a bone graft material;
a bone graft material contained in the cavity;
an implant tool that is selectively attached to the implant device;
a sack that defines a compartment and an opening that provides access to the compartment, wherein the compartment is adapted to contain the implant device with bone graft in the cavity and wherein the opening is adapted to allow the implant tool to extend out of the sack when the implant device is in the compartment; and
a pre-attached closing member that surrounds the opening and is adapted to selectively vary the size of the opening between a size that tightly fits around the implant tool and a size that closes the opening.

14. The system of claim 13, wherein the sack is constructed from a bioabsorbable material.

15. The system of claim 13, wherein the sack is a mesh.

16. The system of claim 13, wherein the mesh is constructed from a suture material.

17. The system of claim 13, wherein the sack is adapted to contain the bone graft material.

18. The system of claim 13, wherein the pre-attached closing member is a length of suture configured as a purse string.

19. The system of claim 13, wherein the implant device is an interbody cage adapted for use to fuse two vertebrae together.

20. The system of claim 13, wherein the interbody cage defines a hole that extends through the interbody cage, wherein the hole is filled with the bone graft material.
